(19) United States
(12) Plant Patent Application Publication
(10) Pub. No.: US 2004/0205872 P1
(43) Pub. Date: Oct. 14, 2004

(54) VARIETY OF BEGONIA PLANT NAMED 'BBVERONICA'

(75) Inventor: Tobias Gunter Dummen, Rheinberg (DE)

Correspondence Address: Russell D. Orkin, 700 Koppers Building, 436 Seventh Avenue, Pittsburgh, PA, 15219-1818, US

(73) Assignee: Begonia Breeders Association B.V.

(21) Appl. No.: 10/409,943

(22) Filed: Apr. 9, 2003

(51) Int. Cl. A01H 5/00

(52) U.S. Cl. PLT/349

(57) ABSTRACT

A new and distinct early flowering Begonia plant with dark red colored flowers.

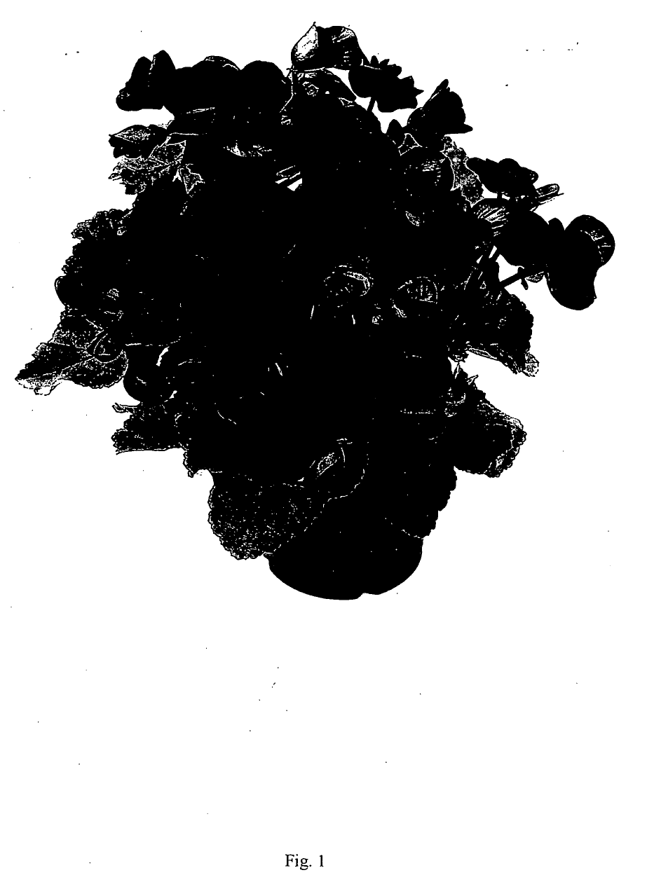

### BOTANICAL CLASSIFICATION

Begonia elatior

### VARIETAL DENOMINATION

‘BBVERONICA’

### BACKGROUND OF THE INVENTION

The present invention comprises a new and distinct cultivar of Begonia plant known by the varietal name ‘BBVERONICA’. The new variety was discovered in 1999 in a selected breeding program in Aalsmeer, Netherlands. The new variety is a mutation of ‘Line’ (unpatented parent). The new variety has a shape and vigor similar to its parent, but differs in flower color and rate of opening from its parent. The new variety was first asexually reproduced in 1999 by cuttings in Aalsmeer, Netherlands. The new variety has been trial and field tested at Aalsmeer and has been found to retain its distinctive characteristics and remain true to type through successive propagations.

The following traits distinguish ‘BBVERONICA’ as a new and distinct cultivar:

1. The flower color is dark red.

2. The flower exhibits better opening.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth characteristics of the new cultivar. The data which defines these characteristics were collected by asexual reproductions by cuttings carried out in Aalsmeer, Netherlands. Plants for the description were 12 weeks old and in 13 cm containers. The color readings were taken in a shaded greenhouse under natural light in the summer. Color references are primarily to the 2001 R.H.S. Colour Chart of The Royal Horticultural Society of London.

### PLANT

Form: Upright; compact.

Height:

Media surface to top of leaves.—18 cm.

Media surface to top of flowers.—22 cm.

Width: 25 cm.

Lateral braches:

Number.—4-5.

Color.—145A.

Foliage:

Arrangement.—Alternate.

Quantity.—6 per lateral branch.

Length.—9 cm.

Width.—8 cm.

Shape.—Asymmetrical ovate.

Apex.—Acuminate.

Base.—Oblique.

Margin.—Crenate.

Texture.—Smooth-velvety.

Color.—N139.

Petiole.—Diameter: 4 mm. Color: 145A.

Stipule.—Quantity: 2 per leaf, 1 cm. Shape: Asymmetrical; pointed. Apex: Acute. Base: Straight. Margin: Entire. Color: Upper surface: N139B. Lower surface: N139C.

Venation.—Upper surface: N139B. Lower surface: N139C.

### FLOWER

Natural flowering season: Year round; continuous.

Flowering longevity on the plant: 4 weeks.

Flowers:

Arrangement.—Compound; not aborted.

Cyme diameter.—7-8 cm.

Number of flowers per cyme.—5-6.

Aspect.—Flat to cupped.

Shape.—Oval; round.

Diameter.—5 cm.

Height.—1 cm.

Fragrance.—None.

Number of flowers per plant.—30.

Bud:

Shape.—Oval.

Diameter.—2 cm.

Length.—1.5 cm.

Color.—44A.

Rate of flower opening.—3-4 days.

Petals:

Arrangement.—Double.

Shape.—Rounded; obovate.

Apex.—Rounded.

Base.—Cordate.

Margin.—Entire.

Quantity.—10-18 per flower.

Size.—Length: 3 cm. Width: 4 cm.

Texture.—Smooth.

Color.—Upper surface: 42A. Lower surface: 42B.

Peduncles:

Angle.—30-45° from vertical.

Strength.—Strong.

Length.—5-6 cm.

Color.—30C.

Pedicels:

Angle.—30-45° from vertical.

Strength.—Strong.

Length.—2-3 cm.

Color.—30C.

Bracts:

Arrangement.—Opposite.

Shape.—Broadly cordate.

Apex.—Acute.

Margin.—Entire.

Color.—Upper surface: 41A. Lower surface: 41A.

Reproductive organs: None.

Disease resistance: Resistant to mildew.

Seed production: None.

I claim:

1. A new and distinct variety of Begonia plant substantially as shown and described.
